(19) United States
(12) Patent Application Publication
(10) Pub. No.: US 2026/0041385 A1
(43) Pub. Date: Feb. 12, 2026

(54) MEDICAL WORK SUPPORT SYSTEM, WORK SUPPORT METHOD, AND NON-TRANSITORY RECORDING MEDIUM

(71) Applicants: CANON KABUSHIKI KAISHA, Tokyo (JP); CANON MEDICAL SYSTEMS CORPORATION, Tochigi (JP)

(72) Inventors: SHUNSUKE TSUDA, Tokyo (JP); HIROMI KINEBUCHI, Kanagawa (JP); RISA HAYASHI, Tochigi (JP); HARUKI IWAI, Tochigi (JP)

(21) Appl. No.: 19/285,727

(22) Filed: Jul. 30, 2025

(30) Foreign Application Priority Data
Aug. 6, 2024 (JP) 2024-130109
May 22, 2025 (JP) 2025-085654

(51) Int. Cl. A61B 6/00; A61B 5/00; A61B 6/04; A61B 6/46; G16H 30/20

(52) U.S. Cl. CPC A61B 6/4429; A61B 5/7267; A61B 6/04; A61B 6/4405; A61B 6/461; A61B 6/5217; G16H 30/20

(57) ABSTRACT

Provided is a medical work support system including: an image data acquisition unit which acquires image data obtained through photographing in a state where a subject to be examined is placed on a bed; a placement region acquisition unit which acquires, from the image data, a placement region for placing the subject to be examined; a subject-to-be-examined position data calculation unit which calculates, from the image data, position data on the subject to be examined; and an abnormality determination unit which determines whether an abnormality is present in a position of the subject to be examined based on the placement region acquired by the placement region acquisition unit and the position data on the subject to be examined.

### BACKGROUND

### Field of the Technology

The present disclosure relates to a medical work support system for detecting a subject to be examined who is placed on a bed of a medical imaging apparatus, and a work support method therefor.

### Description of the Related Art

A large-sized medical imaging apparatus that captures a diagnostic image while controlling an orientation of a bed on which a subject to be examined is placed, such as an X-ray diagnostic apparatus, an X-ray computed tomography (CT) apparatus, a magnetic resonance imaging (MRI) apparatus, a positron emission tomography (PET) apparatus, or a single photon emission computed tomography (SPECT) apparatus, includes a large number of movable portions. When operating the medical imaging apparatus, it is required to carefully avoid contact between the movable portions of the medical imaging apparatus and the subject to be examined as well as nearby doctors, nurses, and examination equipment, and hence a thorough safety confirmation measure is desired.

One such safety confirmation measure is a medical work support system that uses cameras installed in an examination room. A plurality of cameras may be installed in order to reduce blind spots. Moving image data acquired from each camera is subjected, by an image processing apparatus such as a computer, to information extraction, composite video creation, and danger level determination so as to obtain information required for safety confirmation, and an operator of the medical imaging apparatus is notified of the obtained information through a monitor near the operator.

In the safety confirmation relating to the subject to be examined, the medical work support system is required to accurately detect body parts of the subject to be examined in order to recognize positions of hands and arms, which tend to come into contact with the movable portions, and recognize facial expressions for prediction of sudden behaviors.

In Japanese Patent No. 7118666, there is disclosed an X-ray diagnostic apparatus having a function of detecting a subject to be examined through use of a differential image between an image of a bed top plate photographed in a state where the subject to be examined is not lying and an image of the bed top plate photographed in a state where the subject to be examined is lying. That is, in Japanese Patent No. 7118666, reference image data is obtained through photographing by a camera in a state where a person is not present, and it is possible to detect a person from a differential image between image data obtained through photographing in a state where a person is present and the reference image data.

In Japanese Patent No. 5959972, there is disclosed an X-ray diagnostic apparatus involving a method of arranging a plurality of cameras, selecting an appropriate one from thereamong, and causing an image acquired by the selected camera to be displayed to an operator of the apparatus, and having a function of switching to an appropriate camera depending on an inclination angle of a movable bed top plate for the purpose of ensuring a field of view of the operator.

In Japanese Patent No. 7118666, there has been a problem in that the reference image data is required to be acquired again due to a change in the reference image data when an illumination device or the camera deteriorates over time or when a medical imaging apparatus is moved.

In Japanese Patent No. 5959972, there has been a problem in that the operator of the apparatus is required to pay attention not only to a camera image but also to an operation of driving the apparatus, resulting in a heavy burden of paying attention to positions of the subject to be examined even when an appropriate image can be displayed.

### SUMMARY

Accordingly, the present disclosure is directed to providing a medical work support system capable of detecting an abnormality in a position of a subject to be examined with a light burden and a simple operation.

According to the present disclosure, there is provided a medical work support system including: an image data acquisition unit which acquires image data obtained through photographing in a state where a subject to be examined is placed on a bed; a placement region acquisition unit which acquires, from the image data, a placement region for placing the subject to be examined; a subject-to-be-examined position data calculation unit which calculates, from the image data, position data on the subject to be examined; and an abnormality determination unit which determines whether an abnormality is present in a position of the subject to be examined based on the placement region acquired by the placement region acquisition unit and the position data on the subject to be examined.

Features of the present disclosure will become apparent from the following description of embodiments with reference to the attached drawings. The following description of embodiments is described by way of example.

### BRIEF DESCRIPTION OF THE DRAWINGS

FIG. 1 is a schematic view for illustrating an example of a configuration of a medical work support system according to an embodiment of the present disclosure.

FIG. 2 is a flow chart for illustrating an example of a processing procedure for a work support method according to a first embodiment.

FIG. 3 is a flow chart for illustrating an example of processing for calculating a top plate region in the work support method according to the first embodiment.

FIG. 4 is a view for illustrating an arrangement example of feature points set in advance.

FIG. 5A is a view for illustrating training data for a deep learning model.

FIG. 5B is a heat map for illustrating the training data for the deep learning model.

FIG. 6 is a heat map for illustrating a result of estimating coordinates of each feature point.

FIG. 7 is a heat map for illustrating a result of estimating the coordinates of each feature point.

FIG. 8 is a graph for showing how to acquire boundary parameters.

FIG. 9 is a flow chart for illustrating an example of processing in a top plate region cut-out step in the work support method according to the first embodiment.

FIG. 10 is a view for illustrating parameters for cutting out the top plate region.

FIG. 11 is a view for illustrating a method of determining whether or not an abnormality is present in the work support method according to the first embodiment.

FIG. 12A is a view for illustrating an example of a method of notifying of a possibility of interference.

FIG. 12B is a view for illustrating the example of the method of notifying of the possibility of interference.

FIG. 13A is a view for illustrating an example of a method of notifying of the possibility of interference in the first embodiment.

FIG. 13B is a view for illustrating the example of the method of notifying of the possibility of interference in the first embodiment.

FIG. 14A is a view for illustrating an example of the method of notifying of the possibility of interference in the first embodiment.

FIG. 14B is a view for illustrating the example of the method of notifying of the possibility of interference in the first embodiment.

FIG. 15A is a view for illustrating a method of determining whether or not an abnormality is present in a modification example of the first embodiment.

FIG. 15B is a view for illustrating the method of determining whether or not an abnormality is present in the modification example of the first embodiment.

FIG. 16A is a view for illustrating an influence of camera parallax.

FIG. 16B is a view for illustrating the influence of the camera parallax.

FIG. 17 is a flow chart for illustrating an example of a processing procedure for a work support method according to a second embodiment.

FIG. 18 is a view for illustrating a method of determining whether or not an abnormality is present in the work support method according to the second embodiment.

### DESCRIPTION OF THE EMBODIMENTS

Now, each configuration of the present disclosure will be described in detail with reference to the drawings through use of exemplary embodiments of the present disclosure. Like elements or corresponding elements are denoted by the same reference numerals in the drawings, and description thereof may be omitted or simplified.

In the following discussion, when references are made to specific directions such as left, right, front, back, up, and down, those directions are to be understood as being described from the perspective of a user facing a system described below during an exemplary operation.

### First Embodiment

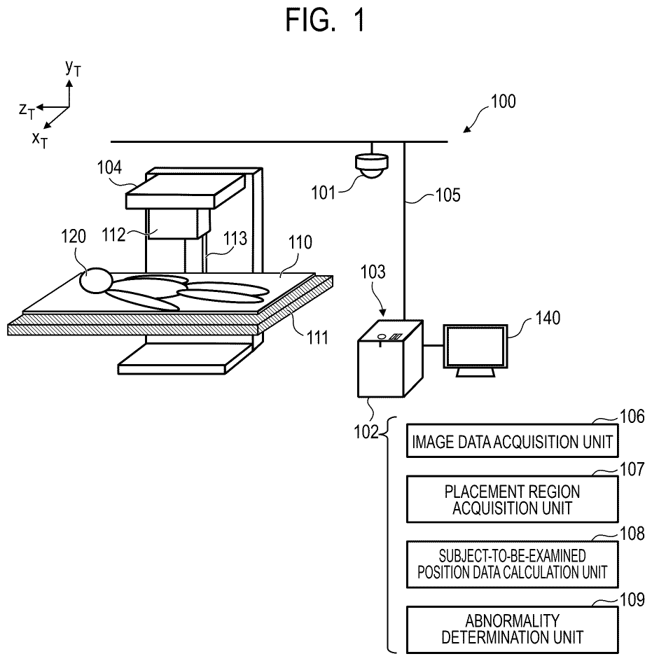

A first embodiment according to the present disclosure is described with reference to FIG. 1 to FIG. 15B. Here, an example of a case in which a medical imaging apparatus is an X-ray diagnostic apparatus is described.

FIG. 1 is a schematic view for illustrating an example of a configuration of a medical work support system according to the first embodiment. A medical work support system 100 according to the present disclosure includes a camera 101, an image processing apparatus 102 included in a personal computer (PC) 103, and a notification apparatus 140 that notifies the user of an abnormality in a position of a subject to be examined.

The camera 101 is fixed to a ceiling of an examination room so as to photograph an environment including a placement region for placing a subject 120 to be examined and the subject 120 to be examined. In this case, the placement region is a region corresponding to a range in which the placed subject 120 to be examined is required to be accommodated, and is typically a region of a top plate 110 or a bed 111. The camera 101 is, for example, an optical camera. A plurality of cameras 101 may be installed in order to reduce blind spots. After the medical work support system 100 is started up, the camera 101, an image processing apparatus 102, and the PC 103 can communicate to/from each other through a network 105.

The image processing apparatus 102 includes an image data acquisition unit 106, a placement region acquisition unit 107, a subject-to-be-examined position data calculation unit 108, and an abnormality determination unit 109.

The image data acquisition unit 106 acquires image data obtained through photographing in a state where the subject 120 to be examined is placed on the bed 111. The placement region acquisition unit 107 acquires the placement region for placing the subject 120 to be examined from the image data acquired by the image data acquisition unit 106. The subject-to-be-examined position data calculation unit 108 calculates position data on the subject 120 to be examined from the image data acquired by the image data acquisition unit 106. Further, the abnormality determination unit 109 determines whether or not an abnormality is present in the position of the subject 120 to be examined based on the placement region acquired by the placement region acquisition unit 107 and the position data on the subject to be examined.

The PC 103 including the image processing apparatus 102 includes a central processing unit (CPU). The CPU performs a predetermined operation in accordance with a program stored in a random access memory (RAM), a read only memory (ROM), a hard disk drive (HDD), or the like provided to the PC 103 or to another apparatus that functions through the network 105. Processing to be performed by the CPU may include not only control of respective functions of the PC 103 but also respective kinds of processing to be performed by the image data acquisition unit 106, the placement region acquisition unit 107, the subject-to-be-examined position data calculation unit 108, and the abnormality determination unit 109. The above-mentioned respective kinds of processing may also be executed by a micro controller unit (MPU) instead of being executed by the CPU.

An example in which the image processing apparatus 102 is included in the PC 103 is described here, but the image processing apparatus 102 may be formed by a workstation, a server, a field programmable gate array (FPGA), an application specific integrated circuit (ASIC), a microcomputer, or the like. The image processing apparatus 102 may also be formed by a tablet PC, a smartphone, or the like that is integrated with the notification apparatus 140.

The notification apparatus 140 notifies the user of the abnormality determined by the abnormality determination unit 109. Specifically, the notification apparatus 140 notifies the user of a determination result regarding a possibility of interference between the subject 120 to be examined and the X-ray diagnostic apparatus 104 obtained by the abnormality determination unit 109. The notification apparatus 140 is, for example, a display for visual display or a speaker for audio notification. The notification apparatus 140 may be any apparatus that can notify the user of the possibility of interference of the subject 120 to be examined. In the first embodiment, an example in which the notification apparatus 140 is a display for visual display is described.

The X-ray diagnostic apparatus 104 having the placement region to be photographed by the camera 101 is described. The X-ray diagnostic apparatus 104 includes the top plate 110, the bed 111, and an X-ray tube 112. It is assumed that, in FIG. 1, a short-side direction of the top plate 110 is an XT direction, a direction perpendicular to a surface of the top plate 110 on which the subject 120 to be examined is to be placed is a y_{T} direction, and a long-side direction of the top plate 110 is a z_{T} direction.

In a major examination such as an upper gastrointestinal examination, the subject 120 to be examined lies on the top plate 110 along the long-side direction (Z_{T} direction) of the top plate 110. The top plate 110 moves on the bed 111 along the X_{T} direction, and the X-ray tube 112 and a support column 113 move along the y_{T} direction and the z_{T} direction. The top plate 110, the bed 111, the X-ray tube 112, and the support column 113 integrally rotate around an axis conforming to the X_{T} direction, and a posture of the subject 120 to be examined can be changed from a lying position (supine position) to a standing position. That is, the placement region for placing the subject 120 to be examined is movable. In this manner, the X-ray diagnostic apparatus 104 has a large number of movable portions, and hence the user operating the X-ray diagnostic apparatus 104 is required to ensure safety by predicting behaviors of the subject 120 to be examined while observing states of the subject 120 to be examined.

A flow of the processing to be performed by the medical work support system 100 is described.

The camera 101 performs photographing at an angle of view including the placement region and the subject 120 to be examined, and transmits acquired camera image data to the image processing apparatus 102 built into the PC 103 on a frame-by-frame basis. The image processing apparatus 102 detects, for example, the top plate 110 serving as the placement region and a head portion of the subject 120 to be examined from the transmitted camera image data, and generates display image data in which the top plate 110 and the subject 120 to be examined are cut out and composited, display image data indicating the possibility of interference between the subject 120 to be examined and the X-ray diagnostic apparatus 104, and the like. The display image data is displayed on the notification apparatus 140 through intermediation of the PC 103 on a frame-by-frame basis.

The user operating the X-ray diagnostic apparatus 104 operates the X-ray diagnostic apparatus 104 while confirming the safety of the subject 120 to be examined by viewing a display screen of the notification apparatus 140.

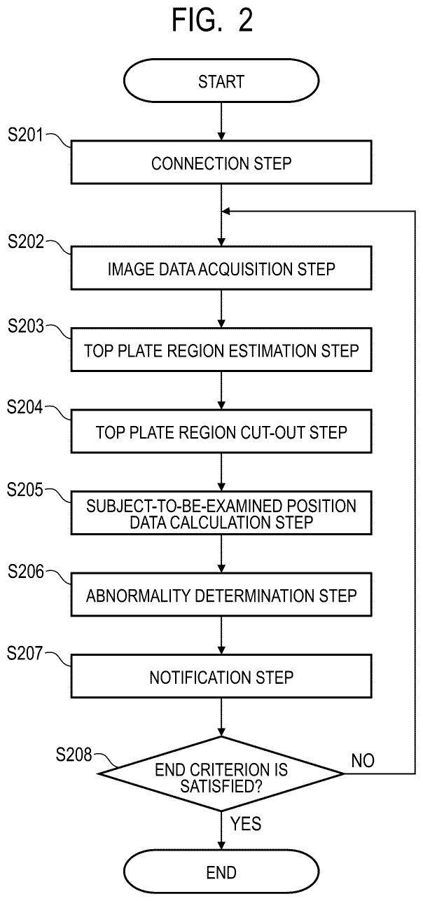

A detailed processing procedure to be performed by the medical work support system 100 is described with reference to FIG. 2. FIG. 2 is a flow chart for illustrating an example of a processing procedure for a work support method in the first embodiment.

The work support method according to the first embodiment includes an image data acquisition step S202, a placement region acquisition step, a subject-to-be-examined position data calculation step S205, and an abnormality determination step S206, and the placement region acquisition step includes a top plate region estimation step S203 and a top plate region cut-out step S204. In the first embodiment, an example in which the work support method further includes a connection step S201, a notification step S207, and an end determination step S208 is described.

The connection step S201 is a step of opening an input stream of the camera image data sent from the camera 101 and an output stream for transmitting the display image data to the PC 103.

The image data acquisition step S202 is a step acquiring, by the image data acquisition unit 106, the most recent camera image data sent from the input stream. That is, the image data acquired by the image data acquisition unit 106 is generated by photographing the environment including the placement region and the subject 120 to be examined by the camera 101.

As described above, the X-ray diagnostic apparatus 104 has a large number of movable portions, and hence there is a risk of contact or pinching when the subject 120 to be examined puts his or her hand outside the top plate 110 or grabs an edge portion of the top plate 110 by his or her fingers while the apparatus is being driven. In order to detect such a situation, in the abnormality determination step S206, the camera image data is used as input to determine whether or not there is a possibility that the subject 120 to be examined may interfere with the X-ray diagnostic apparatus 104.

The image data acquisition step S202, the top plate region estimation step S203, the top plate region cut-out step S204, the subject-to-be-examined position data calculation step S205, and the abnormality determination step S206 are performed by the image processing apparatus 102.

The placement region acquisition step is a step of acquiring the placement region for placing the subject 120 to be examined from the image data. That is, the placement region is the region corresponding to the range in which the placed subject 120 to be examined is required to be accommodated, and the medical work support system 100 detects an abnormality based on whether or not the subject 120 to be examined is accommodated in the placement region. An example in which the placement region acquisition unit 107 identifies a plurality of feature points positioned on a boundary of the placement region from the image data in the placement region acquisition step is described below.

In the first embodiment, an example of a case in which the placement region is a subject-to-be-examined placement surface of the top plate 110 provided on the bed 111 is described. The placement region can also be set to a subject-to-be-examined support surface (surface on which the top plate 110 is provided) of the bed 111.

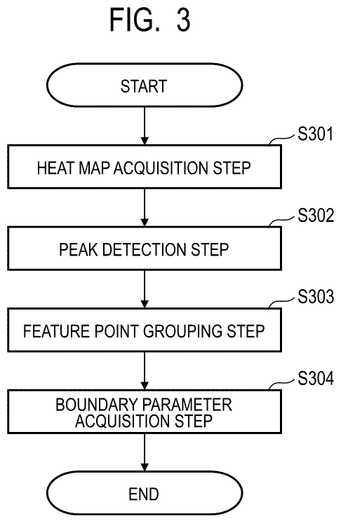

In the top plate region estimation step S203, the placement region acquisition unit 107 acquires parameters that characterize the boundary of the top plate 110 from the camera image data captured into the image processing apparatus 102. A detailed processing procedure for the top plate region estimation step S203 is described with reference to the flow chart of FIG. 3. In the first embodiment, an example including a heat map acquisition step S301 using a machine learning model in order to estimate a top plate region is described.

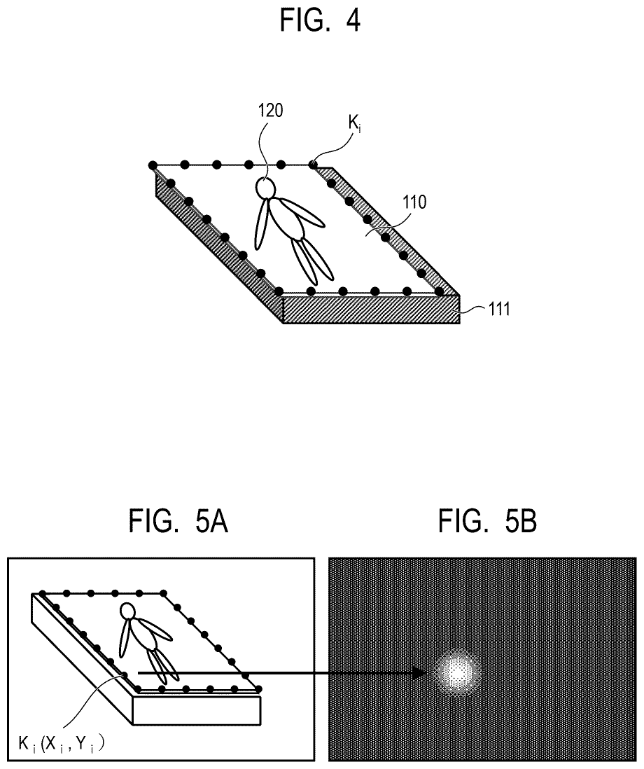

FIG. 4 is a view for illustrating the top plate 110 and the bed 111 of the X-ray diagnostic apparatus 104, the subject 120 to be examined, and a plurality of virtual feature points Kᵢ (K₁, K₂, . . . . K_{N}) that are set in advance at freely-selected positions around the top plate 110 (N is a total number of set feature points). A plurality of black dots drawn around the top plate 110 represent the plurality of feature points Kᵢ.

In the heat map acquisition step S301, coordinate estimation processing for estimating the coordinates of any feature points Kᵢ set around the top plate 110 from the input image is performed. Here, an example in which the placement region acquisition unit 107 identifies a plurality of feature points through use of the machine learning model is described. That is, the coordinates of each feature point Kᵢ can be estimated through use of, for example, a deep learning model that outputs an image such as an autoencoder.

In order for the deep learning model to learn, training data is required. The training data as used herein refers to training image data in which the top plate 110 appears and annotation data indicating coordinates at which the feature points Kᵢ are present in each image. In regard to a method of acquiring the training image data and the annotation data, it is possible to use, for example, computer graphics (CG). The use of CG enables creation of a variety of kinds of training image data as well as accurate acquisition of the coordinates of the feature points Kᵢ on the image.

As another method of obtaining the training data, it is possible to directly determine a position and an orientation of the top plate 110 by obtaining the training image data on the top plate 110 through photographing using a camera and simultaneously attaching freely-selected markers such as AR markers to the top plate 110, to thereby acquire the training data.

Assuming that the image in which the top plate 110 appears has a height H, a width W, and the number C of color channels (1 for a grayscale image and 3 for an RGB color image), the training image data is a data array having a size of H×W×C.

In a case of the training image data having the size described above, the annotation data to be used for training can be, for example, a data array of H×W×N, where N represents the number of channels that is the above-mentioned total number of virtual feature points. That is, the annotation data is a collection of as many two-dimensional arrays as the total number N of the feature points Kᵢ, the two-dimensional arrays cach having the same height and width as those of the training image data. Channels 1 to N of the annotation data represent the positions of feature points K₁ to K_{N}, respectively.

FIG. 5A is a view in which the position of a feature point Kᵢ is displayed by being superimposed on the training image data, and FIG. 5B is a heat map in which a piece of annotation data corresponding to the i-th feature point (channel “i” of the annotation data) is expressed as an image.

When the coordinates of the feature point Kᵢ in the training image data are (xᵢ, yᵢ), the channel “i” of the annotation data is assumed to be an array (heat map) of numerical values that decay in accordance with a Gaussian distribution with the coordinates (xᵢ, yᵢ) being set as a peak. The heat map is a two-dimensional array including values of from 0 to 1 with the peak value being 1 and the value at a position sufficiently far from the peak being 0. In this case, the numerical values included in the heat map correspond to an existence probability of the feature point Kᵢ. The annotation data can be said to be a collection of such heat maps centered on the positions of the feature points Kᵢ.

Providing the annotation data as such a heat map as described above enables improvement in training accuracy of the deep learning model.

The height and width of an annotation data array size can be changed to any size as long as the number N of channels is fixed. For example, setting the annotation data array size to H/2, W/2, and N enables the number of parameters of the deep learning model to be reduced compared to when the height and width are set the same as those of the training image data. Thus, an effect of shortening a time required for training the model and a calculation time during inference is expected to be produced.

In the heat map acquisition step S301, for each feature point Kᵢ (i=1 to N), the existence probability of the feature point Kᵢ at each set of coordinates is acquired in the form of a heat map. This results in acquisition of the data array of H×W×N. In this case, the data array of the channel “i” (i=1 to N) is the heat map representing the existence probability (0 to 1) of the feature point Kᵢ, and as the value at each set of coordinates in the data array becomes larger, the probability that the feature point is present at that set of coordinates becomes higher.

As an image for estimating the existence probability of the feature point Kᵢ in the heat map acquisition step S301, the camera image acquired in the image data acquisition step S202 can be used. In another case, an image obtained by subjecting the camera image to any image processing including a linear processing filter such as edge enhancement or a nonlinear processing filter such as a median noise reduction filter may be used.

Subsequently, in a peak detection step S302, peak detection is performed from cach channel by any method to estimate the coordinates of the feature point Kᵢ on the image.

The feature points the coordinates of which can be estimated by the peak detection may be some of the N feature points Kᵢ (i=1 to N). Specifically, for example, it may not be possible to predict the coordinates of a feature point positioned on a back side of an object when viewed from the camera 101 or a feature point hidden by another object such as a body of the subject 120 to be examined or a nearby doctor or nurse.

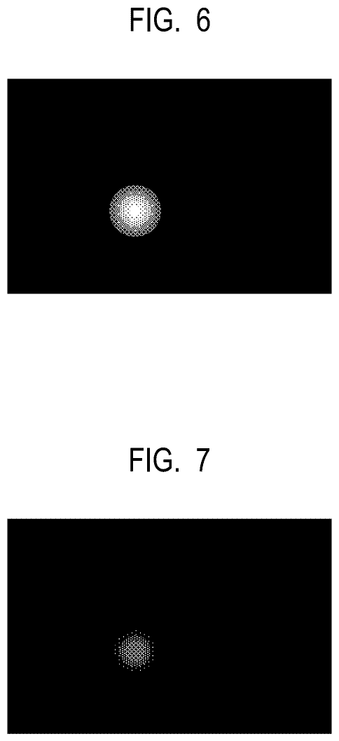

FIG. 6 shows a heat map of a feature point having a high degree of confidence, and FIG. 7 shows a heat map of a feature point having a low degree of confidence.

In the peak detection in the peak detection step S302, it is possible to set a threshold value for discriminating whether or not the value indicated by the coordinates is a peak. For example, it is assumed that the threshold value of the degree of confidence for determining a peak is 0.4. It is also assumed that, as a result of the peak detection, a coordinate value indicating the peak value is 0.9 in FIGS. 6 and 0.3 in FIG. 7. At this time, the coordinates of the feature point of that index are estimated in the case of FIG. 6, but the coordinates of the feature point of that index are not estimated in the case of FIG. 7.

In the following description, the position of the feature point Kᵢ estimated from the image is referred to as an estimated feature point Pⱼ (1≤j≤N), and a set of indices “j” of successfully estimated feature points is represented by J.

In a feature point grouping step S303, processing for grouping the set J of successfully inferred feature points is performed for the peaks detected from the heat maps in the peak detection step S302. The top plate 110 has a substantially rectangular shape, and hence it is possible to define which of the four sides each of the feature points Kᵢ set around the top plate 110 belongs to. When the feature points Kᵢ are present at the four corners of the rectangle, each of the feature points Kᵢ can be handled as belonging to both of two intersecting sides.

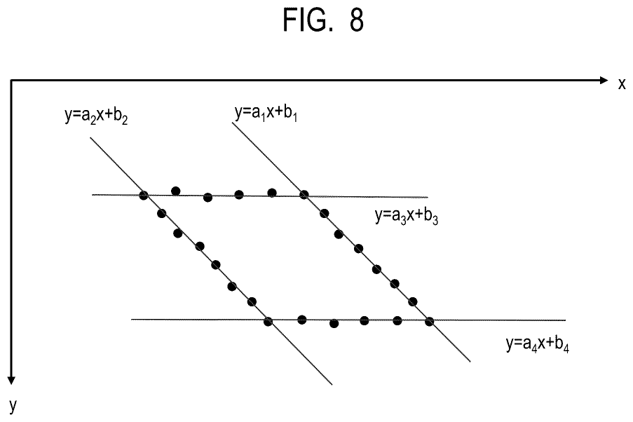

In a boundary parameter acquisition step S304, parameters indicating the four sides that characterize the boundary of the top plate 110 are acquired. For example, as shown in FIG. 8, the four sides of the top plate 110 can each be approximated by a linear expression in the form of y=ax+b in a coordinate system within the camera image. The parameters (a, b) can be obtained by a least-square method through use of sets of coordinates of a plurality of feature points Kᵢ that belong to the same group based on the grouping performed in the feature point grouping step S303. In a case of thus approximating lines of the boundary of the top plate 110 by linear expressions, (2 parameters)×(4 sides)=8 parameters are estimated.

When the four sides of the top plate 110 are viewed in the camera image, the four sides are not actually straight lines due to an influence of distortion (distortion aberration) caused by aberration of a camera lens. For that reason, a more accurate boundary of the top plate 110 can be obtained by approximating by a polynomial of degree two or higher in the boundary parameter acquisition step S304. In order to avoid overfitting, it is desired to limit the degree of the polynomial to at most three.

As another method, it is also possible to correct the distortion aberration in a camera image by estimating internal parameters by camera calibration. For example, there is known a method in which a checkerboard or the like is photographed from a plurality of angles to estimate internal parameters from corresponding points in a three-dimensional space and in a camera image. There is also known another method in which a straight line within the camera image is detected to estimate a distortion coefficient that minimizes the distortion of the straight line. When a camera image in which distortion aberration is corrected by the internal parameters acquired in advance is obtained, the four sides of the top plate 110 are approximately straight lines on the camera image, and hence physically appropriate parameters can be estimated by approximating by linear expressions.

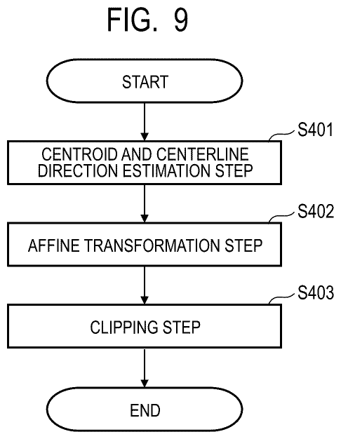

In the top plate region cut-out step S204, the placement region acquisition unit 107 performs processing for cutting out a region including the top plate 110 as a rectangular region of interest (ROI). A detailed flow of the top plate region cut-out step S204 is illustrated in FIG. 9.

In a centroid and centerline direction estimation step S401, a centroid position of the top plate 110 and a direction vector (hereinafter referred to as “centerline direction”) corresponding to the long-side direction of the top plate 110 in the camera image are estimated. The centroid position of the top plate 110 can be estimated by, for example, obtaining average coordinates of the plurality of feature points Kᵢ that have been obtained in the peak detection step S302. When the number of the estimated plurality of feature points Kᵢ is M, a centroid “w” is given by Expression (1) through use of coordinates xᵢ (i=0, 1, . . . M−1).

$$w = {\sum }_{i = 0}^{M - 1} {x}_{i} / M \qquad ( 1 )$$

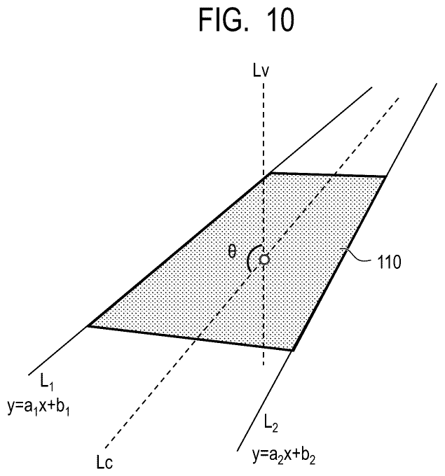

A definition of the centerline is described with reference to FIG. 10. FIG. 10 is a view for illustrating the top plate 110 in the camera image and straight lines L1 and L2 including approximated straight lines corresponding to the long sides of the top plate 110. As illustrated in FIG. 10, when inclinations of the straight lines L1 and L2 including the long sides are a₁ and a₂, respectively, an inclination a_{c} of a centerline Lc is defined as Expression (2).

$${a}_{c} = ( {a}_{1} + {a}_{2} ) / 2 \qquad ( 2 )$$

Subsequently, in an affine transformation step S402, affine transformation is performed on the coordinates of the feature points Kᵢ. From the inclination a_{c} of the centerline Lc defined by Expression (2), an angle θ between the upward vertical direction of the image and Lc is given by Expression (3).

$$θ = arctan ( {a}_{c} ) \qquad ( 3 )$$

Through use of the xᵢ and θ obtained above, it is possible to obtain an affine transformation matrix A having 3 rows and 3 columns as shown in Expression (4) such that the centerline Lc becomes vertical around the centroid “w” of the top plate 110.

$$( \begin{matrix}{x}^{′} \\ {y}^{′} \\ 1\end{matrix} ) = A ( \begin{matrix}x \\ y \\ 1\end{matrix} ) \qquad ( 4 )$$

In Expression (4), (x, y) are coordinates before transformation, and (x′, y′) are coordinates subjected to the affine transformation.

In a clipping step S403, a region including the top plate 110 is clipped as the placement region from the image after the affine transformation. A size of a clipping region can be determined by, for example, transforming the sets of coordinates of the plurality of feature points Kᵢ by Expression (4) and specifying such a rectangle as to include all the sets of coordinates.

In the subject-to-be-examined position data calculation step S205, the subject-to-be-examined position data calculation unit 108 calculates the position data on the subject 120 to be examined on the top plate 110. The position data on the subject 120 to be examined refers to, for example, coordinates of anatomical body parts (landmarks) of the subject 120 to be examined. The landmarks may cover the entire body of the subject 120 to be examined, or may be extracted as positions of parts of the body of the subject 120 to be examined. The landmarks may also be extracted as detailed posture information on the subject 120 to be examined, the detailed posture information being generated by identifying the respective landmarks of the subject 120 to be examined and combining the respective landmarks.

Examples of a method of calculating subject-to-be-examined position data include a method using a machine learning model. Specifically, a trained deep neural network (hereinafter referred to as “DNN”) can be used as the machine learning model. As the trained DNN, it is possible to use such a model as to output positions of body parts (such as hands and feet) of interest of the subject 120 to be examined as bounding box or binary mask information, or a model that outputs a plurality of sets of landmark coordinates of the subject 120 to be examined and the posture information thereon.

The subject-to-be-examined position data is obtained in the coordinate system of the image obtained by clipping the top plate region, and hence Expression (5) is used to transform the subject-to-be-examined position data into the coordinate system before the clipping and the affine transformation.

$$( \begin{matrix}x \\ y \\ 1\end{matrix} ) = {A}^{- 1} ( \begin{matrix}{x}^{′} \\ {y}^{′} \\ 1\end{matrix} ) \qquad ( 5 )$$

In Expression (5), A⁻¹ is the inverse matrix of the affine transformation matrix A, and (x, y) are coordinates obtained by performing the inverse affine transformation on (x′, y′).

In the abnormality determination step S206, the abnormality determination unit 109 determines whether or not an abnormality is present in the position of the subject 120 to be examined based on the placement region acquired in the placement region acquisition step and the position data on the subject 120 to be examined. Specifically, in the abnormality determination step S206, it is possible to use the position data on the subject 120 to be examined on the top plate 110 which has been obtained in the subject-to-be-examined position data calculation step S205 and the boundary parameters of the top plate 110 which have been obtained in the boundary parameter acquisition step S304. Then, whether or not each body part of the body of the subject 120 to be examined is inside the top plate 110 can be determined as presence or absence of an abnormality based on the position data and the boundary parameters.

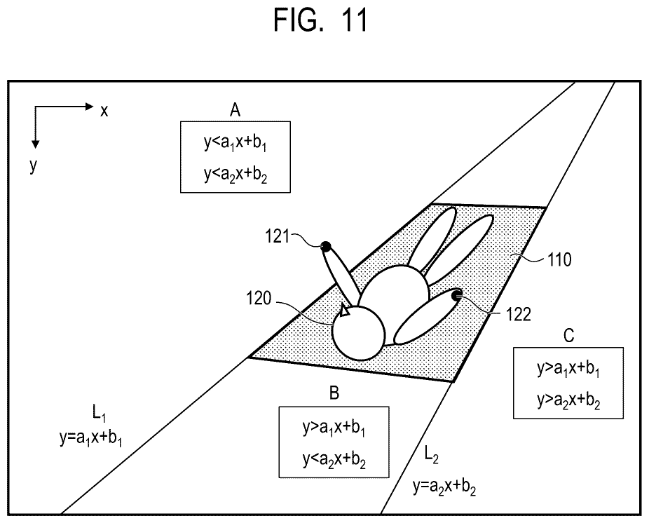

FIG. 11 is a view for illustrating a method of determining whether or not an abnormality is present in the first embodiment. FIG. 11 is an illustration of a state in which the subject 120 to be examined is lying on his or her back on the top plate 110, and a point 121 and a point 122 are landmarks indicating respective positions of the left hand and the right hand of the subject 120 to be examined which have been obtained in the subject-to-be-examined position data calculation step S205.

In FIG. 11, the right hand of the subject 120 to be examined is inside the region of the top plate 110, while the left hand is outside the region of the top plate 110. When the image is divided into three regions A, B, and C as illustrated in FIG. 11 by the straight lines L1 and L2 indicating the boundary of the top plate 110, the coordinates of a point inside each region satisfy the following expressions. The positive directions of the x-axis and the y-axis are the rightward direction and the downward direction of the image, respectively.

$$y < {a}_{1} x + {b}_{1} and y < {a}_{2} x + {b}_{2} \qquad Region A$$

$$y > {a}_{1} x + {b}_{1} and y < {a}_{2} x + {b}_{2} \qquad Region B$$

$$y > {a}_{1} x + {b}_{1} and y > {a}_{2} x + {b}_{2} \qquad Region C$$

In FIG. 11, the point 121 indicating the left hand of the subject 120 to be examined is in the region A, and the point 122 indicating the right hand is in the region B. That is, assuming that the coordinates of the point 121 are (xₗ, yₗ) and the coordinates of the point 122 are (xᵣ, yᵣ), the following hold.

$${y}_{1} < {a}_{1} {x}_{1} + {b}_{1} and {y}_{1} < {a}_{2} {x}_{1} + {b}_{2} \qquad Point 121$$

$${y}_{r} > {a}_{1} {x}_{r} + {b}_{1} and {y}_{r} < {a}_{2} {x}_{r} + {b}_{2} \qquad Point 122$$

In this manner, the abnormality determination unit 109 performs region determination using the boundary parameters in the image for the coordinates of interest, and determines whether or not each body part of the body of the subject 120 to be examined is in a region that may interfere with the apparatus.

Specifically, for example, the abnormality determination unit 109 may be configured to determine which of inside or outside of the placement region each of sets of coordinates (for example, the point 121 and the point 122 in the above-mentioned example) indicating the parts of the body of the subject 120 to be examined based on the position data on the subject 120 to be examined is. The abnormality determination unit 109 may, for example, be further configured to calculate, when a set of coordinates indicating a part of the body of the subject 120 to be examined based on the position data on the subject 120 to be examined is outside the placement region, a distance between the set of coordinates and the placement region.

In the notification step S207, the notification apparatus 140 notifies the user of the abnormality determined in the abnormality determination step S206. Specifically, when a certain body part of the body of the subject 120 to be examined is in a region in which there is a possibility that the certain body part may interfere with the X-ray diagnostic apparatus 104, the notification apparatus 140 notifies the user operating the X-ray diagnostic apparatus 104 to that effect.

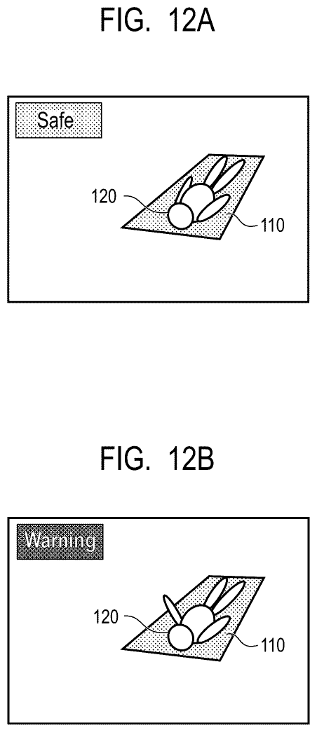

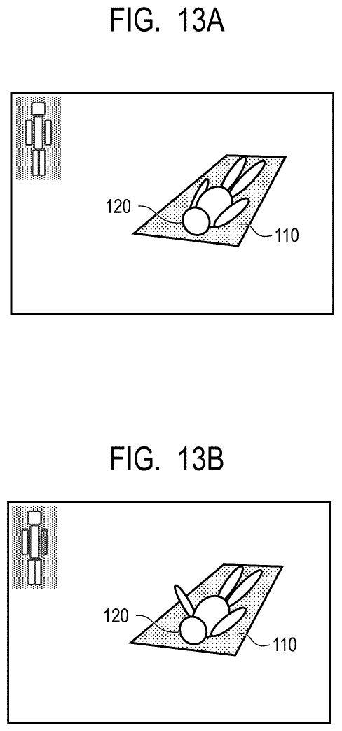

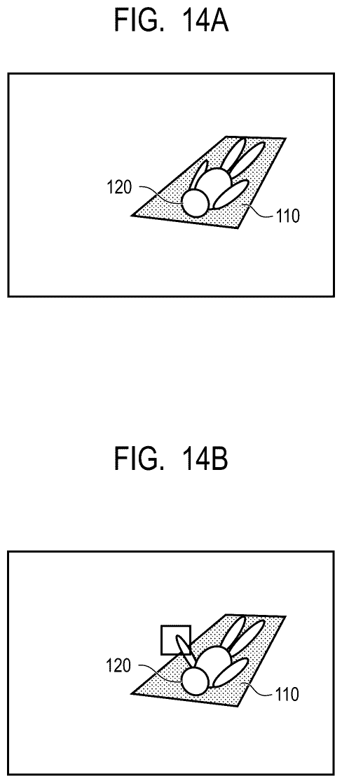

FIG. 12A to FIG. 14B are views for illustrating examples of a method of notifying of the possibility of interference through use of the camera image. FIG. 12A, FIG. 13A, and FIG. 14A are all illustrations of images displayed on the notification apparatus 140 in a state where there is no possibility of interference. Meanwhile, FIG. 12B, FIG. 13B, and FIG. 14B are all illustrations of images displayed on the notification apparatus 140 in a state where there is a possibility of interference due to the subject 120 to be examined having his or her left hand outside the top plate 110 (state in which the notification is performed).

FIG. 12A and FIG. 12B are views for illustrating an example of a case of performing the notification of an abnormality by displaying a warning in the upper left of the screen of the notification apparatus 140. In the example illustrated in FIG. 12A and FIG. 12B, the notification apparatus 140 displays “Safe” as illustrated in FIG. 12A when the subject 120 to be examined has no possibility of interference, and displays “Warning” as illustrated in FIG. 12B when the subject 120 to be examined has the possibility of interference.

FIG. 13A and FIG. 13B are views for illustrating an example of performing the notification of an abnormality by displaying a warning on a pictogram displayed in the upper left of the screen of the notification apparatus 140. The notification apparatus 140 is configured to display, when the subject 120 to be examined has no possibility of interference, an image in which pictograms representing respective body parts of the subject 120 to be examined are uniformly superimposed on an image corresponding to the image data as illustrated in FIG. 13A. The notification apparatus 140 is further configured to display a body part of the subject 120 to be examined which corresponds to the abnormality determined by the abnormality determination unit 109 in an emphasized manner on the corresponding pictogram. That is, specifically, for example, under the state in which the subject 120 to be examined has his or her left hand outside the top plate 110, the notification apparatus 140 displays a part corresponding to the left hand of the subject 120 to be examined by changing a color of the part as illustrated in FIG. 13B.

The notification apparatus 140 may be configured to display an image in which an effect for distinguishably emphasizing a body part of the subject 120 to be examined which corresponds to the abnormality determined by the abnormality determination unit 109 is superimposed on an image corresponding to the image data.

FIG. 14A and FIG. 14B are views for illustrating an example of displaying a possible interference body part of the subject 120 to be examined appearing on the notification apparatus 140 by using a bounding box to emphasize the possible interference body part. When the subject 120 to be examined has no possibility of interference, the camera image is displayed as is on the notification apparatus 140 as illustrated in FIG. 14A. Meanwhile, when the subject 120 to be examined has the possibility of interference, the bounding box is displayed by being overlaid on the left hand of the subject 120 to be examined on the notification apparatus 140 as illustrated in FIG. 14B.

The apparatus or method for notifying the user in the above-mentioned notification step S207 is merely an example, and it is possible to use another method of notifying of the state in which the subject 120 to be examined has the possibility of interference. For example, when the subject 120 to be examined has the possibility of interference, a speaker (not shown) externally connected to the PC 103 may be used as a notification apparatus to notify the user of the possibility of interference.

In the end determination step S208, it is determined whether or not to end the operation of the medical work support system 100 in accordance with a predetermined criterion. When it is determined in the end determination step S208 that the criterion for the end is not satisfied, the process returns to the image data acquisition step S202 to repeatedly perform the steps up to the notification step S207.

That is, until the criterion for the end is satisfied, the camera 101 can photograph the environment including the placement region and the subject 120 to be examined a plurality of times over time. Then, the image data acquisition unit 106 can sequentially acquire the image data that has been obtained through photographing by the camera 101, and the abnormality determination unit 109 can sequentially determine whether or not an abnormality is present in the position of the subject 120 to be examined in parallel with the photographing being performed over time by the camera 101.

When it is determined in the end determination step S208 that the criterion for the end is satisfied, the work support method according to the first embodiment is ended.

The criterion for determining the end can be appropriately determined without any particular restrictions, and may be, for example, presence or absence of an end instruction from the user. In this case, the processing for the notification of the possibility of interference may be stopped in accordance with the instruction from the user. In another example, coordination with the driving of the X-ray diagnostic apparatus 104 may be implemented, and the end can also be determined based on whether the driving of the X-ray diagnostic apparatus 104 is turned on or off.

The configuration example of the X-ray diagnostic apparatus 104 and the medical work support system 100 described in the first embodiment can also be used to implement the work support method according to the present disclosure in other forms, and the medical work support system according to the present disclosure is not limited to the above. For example, the camera 101 may be fixed to the X-ray diagnostic apparatus 104, or may be installed on a tripod or the like. The example of using the X-ray diagnostic apparatus as the medical imaging apparatus has been described in the first embodiment, but any medical imaging apparatus in which an apparatus main body and the subject 120 to be examined move relatively, such as a CT apparatus or an MRI apparatus, may be used.

As described above, with the medical work support system 100 according to the first embodiment, it is possible to determine whether there is a possibility of interference of the subject 120 to be examined with the X-ray diagnostic apparatus 104 and notify the user thereof with a light burden and a simple operation. For example, the medical work support system 100 identifies the placement region and the subject-to-be-examined position based on the same camera image data obtained during an examination through photographing in a state where the subject to be examined is placed on a bed. This enables the medical work support system 100 to determine whether there is a possibility of interference that is the abnormality in the position of the subject 120 to be examined and notify the user thereof without increasing work of acquiring reference image data in advance.

### First Modification Example of First Embodiment

In the first embodiment described above, the existence probability of each feature point is estimated through use of the machine learning model in the top plate region estimation step S203 of FIG. 2, but the present disclosure can also be implemented by other methods. For example, it is possible to detect the top plate region by a method other than the method using machine learning by attaching AR markers, color markers, or the like to the four corners of the top plate 110 and predetermined positions around the top plate 110 and detecting positions of the markers from an acquired camera image.

### Second Modification Example of First Embodiment

In the top plate region estimation step S203 of FIG. 2, a pixel region corresponding to the top plate 110 (hereinafter referred to as “top plate pixel region”) can be detected from a camera image data by a DNN. In this case, as a result of the detection, a binary image (hereinafter referred to as “top plate mask”) in which a value of pixels determined to correspond to the top plate 110 is set to 1 and a value of the other pixels is set to 0 is output.

In the subject-to-be-examined position data calculation step S205, it is also possible to perform processing for detecting a pixel region corresponding to the subject 120 to be examined from the camera image data by a DNN. In this case, as a result of the detection, a binary image (hereinafter referred to as “subject-to-be-examined mask”) in which a value of pixels determined to correspond to the subject 120 to be examined is set to 1 and a value of the other pixels is set to 0 is output.

In the abnormality determination step S206, the camera image obtained in the image data acquisition step S202, the top plate mask, and the subject-to-be-examined mask are input to determine whether there is a possibility that the subject 120 to be examined may interfere with the X-ray diagnostic apparatus 104.

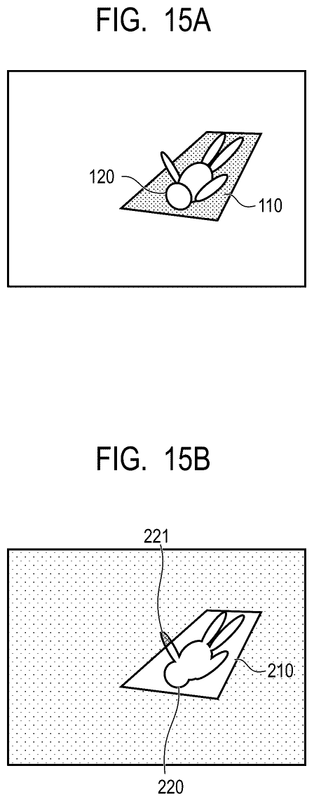

FIG. 15A is a view for illustrating an example of the camera image, and FIG. 15B is a view for illustrating a top plate mask 210 and a subject-to-be-examined mask 220 generated from the camera image illustrated in FIG. 15A. A region 221 included in FIG. 15B represents a region inside the subject-to-be-examined mask 220 and outside the top plate mask 210. In the abnormality determination step S206, it is possible to calculate the area (number of pixels) of the region 221 and determine, when the calculated value is larger than a certain threshold value, that there is a possibility of interference.

### Second Embodiment

A second embodiment according to the present disclosure is described.

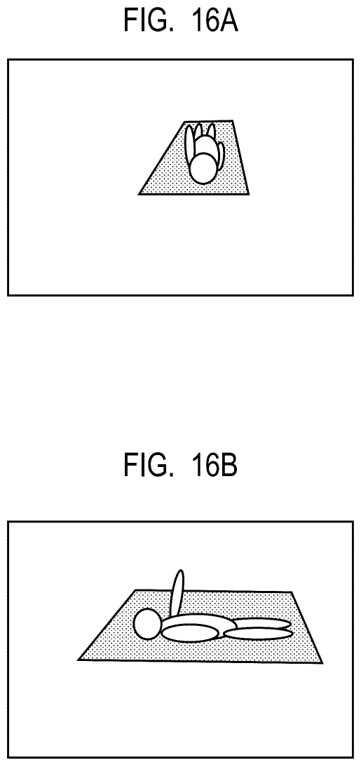

An image photographed by a single camera 101 is influenced by parallax due to perspective projection. FIG. 16A and FIG. 16B are views for illustrating how a posture of the subject 120 to be examined who is lying on the top plate 110 with his or her left hand stretched out in front of his or her body is photographed by cameras at different angles. In the example of the image illustrated in FIG. 16A, the left arm of the subject 120 to be examined is inside the region of the top plate 110, while in the example of the image illustrated in FIG. 16B, the left arm of the subject 120 to be examined appears to be outside the region of the top plate 110. Assuming that the camera 101 is installed at a position at which the image illustrated in FIG. 16B is photographed, it is conceivable that the left arm of the subject 120 to be examined may be determined to be outside the top plate 110 in the abnormality determination step S206 and the user may be notified of an alert.

In the second embodiment, the presence or absence of an abnormality is determined through use of three-dimensional position data on the top plate 110 and the subject 120 to be examined with a camera position being used as a reference.

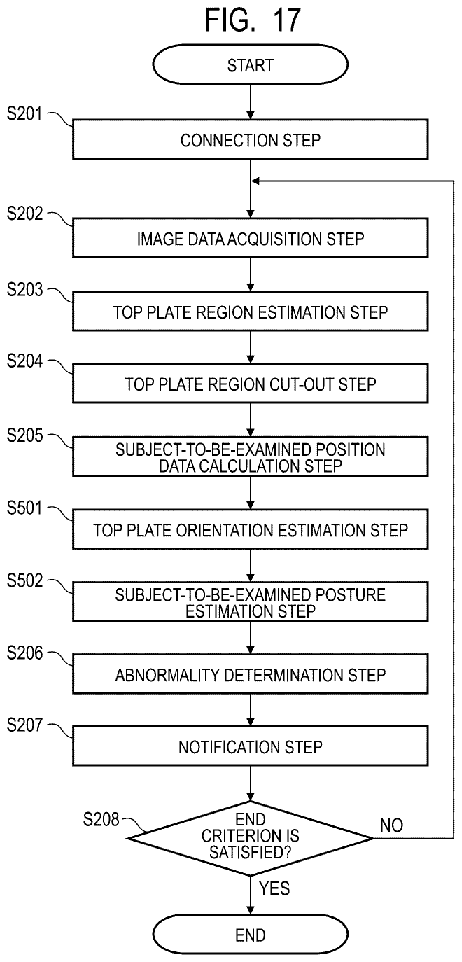

FIG. 17 is a flow chart for illustrating a flow of a work support method according to the second embodiment. The connection step S201 to the top plate region cut-out step S204, the notification step S207, and the end determination step S208 are the same as those in the first embodiment, and hence description thereof is omitted.

In a top plate orientation estimation step S501, the placement region acquisition unit 107 estimates the position and the orientation of the top plate 110 in a coordinate system that uses the camera 101 as a reference. In the PC 103, three-dimensional coordinate information on each feature point Kᵢ is stored in a memory (not shown). The three-dimensional coordinate information on each feature point Kᵢ is a unique index i (i=1 to N) of each feature point Kᵢ and three-dimensional coordinate information (Xᵢ, Yᵢ, Zᵢ) on each individual point (K₁, K₂, . . . K_{N}) of the feature points Kᵢ (i=1 to N).

In the estimation of the position and the orientation of the top plate 110, the coordinates within the camera image of the estimated feature point Pⱼ (1≤j≤N) obtained in the peak detection step S302 and the three-dimensional coordinate information (Xᵢ, Yᵢ, Zᵢ) of each individual point (K₁, K₂, . . . K_{N}) of the feature points Kᵢ are used.

First, among pieces of coordinate information (Xᵢ, Yᵢ, Zᵢ) of the feature points Kᵢ, pieces of information corresponding to the set J of indices “j” of the successfully estimated feature points are read onto the work memory of the PC 103. The position and orientation of the top plate 110 can be estimated by solving a Perspective-n-Point Problem (PnP problem) to obtain external parameters (rotation vector and translation vector) of the camera 101. It is also possible to use in combination a more advanced algorithm relating to the PnP problem or an algorithm that removes outliers, such as random sample consensus (RANSAC).

In the top plate orientation estimation step S501, the position and the orientation of the top plate 110 may be obtained from drive information on the apparatus. The X-ray diagnostic apparatus 104 can be configured to be capable of communicating to/from the medical work support system 100 through the network 105. The image processing apparatus 102 can use information such as an orientation control command for the bed 111 of the X-ray diagnostic apparatus 104 and an examination order through the network 105.

A rotation axis of the bed 111 and the like are known from the design drawing of the X-ray diagnostic apparatus 104, and hence the position and the orientation of the bed 111 can be directly calculated from the orientation control command. It is possible to calculate the position and the orientation of the top plate 110 in the coordinate system that uses the camera 101 as a reference by measuring in advance a positional relationship between the camera 101 and the X-ray diagnostic apparatus 104.

As described above, in the top plate orientation estimation step S501, the position and the orientation of the top plate 110 are obtained in the coordinate system that uses the camera 101 as a reference. Thus, the placement region acquisition unit 107 acquires the placement region as a three-dimensional range calculated as three-dimensional coordinates in a coordinate system that uses as a reference a position at which the image data has been obtained through photographing.

In the subject-to-be-examined position data calculation step S205, two-dimensional coordinates within the camera image of a plurality of landmarks that are anatomical features of the subject 120 to be examined within the camera image and three-dimensional coordinates indicating a relative positional relationship between the landmarks in the space are calculated.

In a subject-to-be-examined posture estimation step S502, the obtained two-dimensional coordinates and three-dimensional coordinates are used to obtain the position and the posture of the subject 120 to be examined with respect to the camera 101 by solving the PnP problem described above. That is, the three-dimensional coordinates of the landmarks of the subject 120 to be examined in the coordinate system that uses the camera 101 as a reference are obtained.

In the manner described above, the subject-to-be-examined position data calculation unit 108 calculates the position data on the subject 120 to be examined as three-dimensional coordinates in the coordinate system that uses as the reference the position at which the image data has been obtained through photographing.

In the abnormality determination step S206, whether there is a possibility of interference between the subject 120 to be examined and the X-ray diagnostic apparatus 104 is determined based on the position and the orientation of the top plate 110 and the three-dimensional coordinates of the landmarks of the subject 120 to be examined in the coordinate system that uses the camera 101 as a reference.

In the abnormality determination step S206, a mathematical function representing a plane including the boundary of the top plate 110 and being perpendicular to the surface on which the subject 120 to be examined lies and the coordinates of the landmarks of the subject 120 to be examined are used to determine presence or absence of the possibility of interference of the subject 120 to be examined with the X-ray diagnostic apparatus 104.

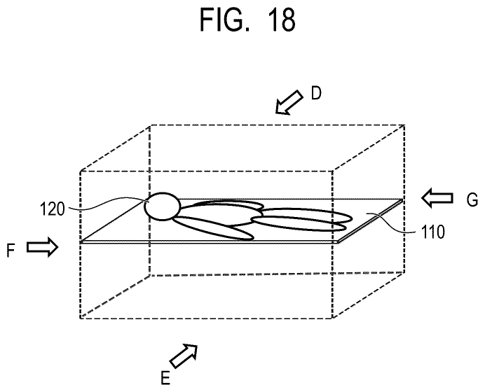

FIG. 18 is a view for illustrating three-dimensional boundary planes D to G of the top plate 110. A set of equations f(x, y, z)=0 that represent the respective boundary planes D to G in the coordinate system that uses the camera 101 as a reference is obtained, to thereby be able to specify four planes surrounding the top plate 110 and conditions for being inside the four planes.

When it is determined in the abnormality determination step S206 that there is a possibility that the subject 120 to be examined may interfere with the X-ray diagnostic apparatus 104, an alert is issued to the user by the notification apparatus 140, an audio notification unit (not shown), or the like in the notification step S207.

As described above, with the medical work support system 100 according to the second embodiment, it is possible to determine whether there is a possibility of interference of the subject 120 to be examined with the X-ray diagnostic apparatus 104 without being influenced by camera parallax, and notify the user of the determination result.

According to the present disclosure, the medical work support system capable of detecting an abnormality in a position of a subject to be examined with a light burden and a simple operation can be provided.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

All of the embodiments described above merely describe embodied examples for carrying out the present disclosure, and thus the technical scope of the present disclosure should not be read as restrictive by the embodiments described above. Specifically, the present disclosure can be carried out in various modes without departing from the technical ideas or main features of the present disclosure. It should be understood that, for example, an embodiment in which a part of the configurations in any one of the embodiments is added to another embodiment, or an embodiment in which a part of the configurations in any one of the embodiments is replaced by a part of the configurations in another embodiment is also an embodiment to which the present disclosure is applicable.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Applications No. 2024-130109, filed Aug. 6, 2024, and No. 2025-085654, filed May 22, 2025, which are hereby incorporated by reference herein in their entirety.

What is claimed is:

1. A medical work support system comprising:
a memory storing instructions, and
at least one processor configured to execute the instructions to:
acquire image data obtained through photographing in a state where a subject to be examined is placed on a bed;
acquire, from the image data, a placement region for placing the subject to be examined;
calculate, from the image data, position data on the subject to be examined; and
determine whether an abnormality is present in a position of the subject to be examined based on the placement region and the position data on the subject to be examined.

2. The medical work support system according to claim 1, further comprising a camera configured to photograph an environment including the placement region and the subject to be examined,
wherein the image data is generated by photographing the environment by the camera.

3. The medical work support system according to claim 2,
wherein the camera is configured to photograph the environment a plurality of times over time, and
wherein the at least one processor is configured to execute the instructions to:
sequentially acquire the image data obtained through photographing by the camera; and
sequentially determine whether an abnormality is present in the position of the subject to be examined in parallel with the photographing of the environment being performed over time by the camera.

4. The medical work support system according to claim 1, further comprising a notification apparatus configured to notify a user of the determined abnormality.

5. The medical work support system according to claim 1, wherein the at least one processor is configured to execute the instructions to identify, from the image data, a plurality of feature points positioned on a boundary of the placement region.

6. The medical work support system according to claim 5, wherein the at least one processor is configured to execute the instructions to identify the plurality of feature points through use of a machine learning model.

7. The medical work support system according to claim 1, wherein the at least one processor is configured to execute the instructions to calculate the position data through use of a machine learning model.

8. The medical work support system according to claim 1, wherein the placement region comprises a subject-to-be-examined placement surface of a top plate provided on the bed.

9. The medical work support system according to claim 1, wherein the placement region comprises a subject-to-be-examined support surface of the bed.

10. The medical work support system according to claim 1, wherein the at least one processor is configured to execute the instructions to determine which of inside or outside of the placement region a set of coordinates indicating a part of a body of the subject to be examined based on the position data on the subject to be examined is.

11. The medical work support system according to claim 1, wherein the at least one processor is configured to execute the instructions to acquire the placement region as a three-dimensional range calculated as three-dimensional coordinates in a coordinate system that uses as a reference a position at which the image data has been obtained through photographing.

12. The medical work support system according to claim 1, wherein the at least one processor is configured to execute the instructions to calculate the position data on the subject to be examined as three-dimensional coordinates in a coordinate system that uses as a reference a position at which the image data has been obtained through photographing.

13. The medical work support system according to claim 1, wherein the placement region is movable.

14. The medical work support system according to claim 4, wherein the notification apparatus is configured to display an image in which pictograms representing respective body parts of the subject to be examined are superimposed on an image corresponding to the image data, and display a body part of the subject to be examined which corresponds to the determined abnormality in an emphasized manner on a corresponding one of the pictograms.

15. The medical work support system according to claim 4, wherein the notification apparatus is configured to display an image in which an effect for distinguishably emphasizing a body part of the subject to be examined which corresponds to the determined abnormality is superimposed on an image corresponding to the image data.

16. The medical work support system according to claim 1, wherein the at least one processor is configured to execute the instructions to calculate, when a set of coordinates indicating a part of a body of the subject to be examined based on the position data on the subject to be examined is outside the placement region, a distance between the set of coordinates and the placement region.

17. The medical work support system according to claim 1, wherein the bed comprises a bed of an X-ray diagnostic apparatus.

18. A work support method comprising:
an image data acquisition step of acquiring image data obtained through photographing in a state where a subject to be examined is placed on a bed;
a placement region acquisition step of estimating, from the image data, a placement region for placing the subject to be examined;
a subject-to-be-examined position data calculation step of calculating, from the image data, position data on the subject to be examined; and
an abnormality determination step of determining whether an abnormality is present in a position of the subject to be examined based on the placement region acquired in the placement region acquisition step and the position data on the subject to be examined.

19. A non-transitory recording medium having stored thereon a program for causing a computer to execute each step of the work support method of claim 18.
